(19) United States
(12) Patent Application Publication
(10) Pub. No.: US 2010/0016736 A1
(43) Pub. Date: Jan. 21, 2010

(54) Estimating Aortic Blood Pressure from Non-Invasive Extremity Blood Pressure

(75) Inventors: Jin-Oh Hahn, Vancouver (CA); Andrew T. Reisner, Newton, MA (US); Haruhiko H. Asada, Lincoln, MA (US)

Correspondence Address: Sunstein Kann Murphy & Timbers LLP, 125 SUMMER STREET, BOSTON, MA, 02110-1618, US

(73) Assignee: MASSACHUSETTS INSTITUTE OF TECHNOLOGY, Cambridge, MA (US)

(21) Appl. No.: 12/501,569

(22) Filed: Jul. 13, 2009

Related U.S. Application Data
(60) Provisional application No. 61/081,185, filed on Jul. 16, 2008.

(51) Int. Cl. A61B 5/021

(52) U.S. Cl. 600/485

(57) ABSTRACT

Methods and a computer program product for using a circulatory measurement on an extremity of a particular subject to derive an aortic blood pressure for that subject. A model is constructed that maps a peripheral cardiovascular waveform to a central cardiovascular waveform on the basis of a plurality of model parameters. A time record is obtained using a non-invasive blood pressure sensor disposed at a solitary position periphery of the cardiovascular system of a subject. The time record is then transformed to obtain a plurality of test central blood pressure waves, with a single test central blood pressure wave is based on each of a plurality of sets of values of the model parameters. An optimum set of values of the model parameters is then selected, based on a specified criterion applied to the plurality of test central blood pressure waves, so that the aortic circulatory waveform of the subject can be obtained.

The present application claims priority from U.S. Provisional Patent Application Ser. No. 61/081,185, filed Jul. 16, 2008, and incorporates that application by reference herein.

### TECHNICAL FIELD

The present invention relates to methods for employing a peripherally derived circulatory waveform to infer features of a patient's core circulatory function, with the methods, more particularly, based on derived patient-specific model parameters.

### BACKGROUND OF INVENTION

While central cardiovascular (CV) signals, such as aortic blood pressure and flow, are generally more informative about cardiac dynamics and global circulation than peripheral cardiovascular signals, the acquisition of central cardiovascular signals typically entails invasive procedures (such as pulmonary artery or central aortic catheterization) that are relatively costly, uncomfortable and risky. Moreover, peripheral circulatory measurements, e.g., arterial blood pressure measured at distal extremity locations, cannot be used as a direct surrogate for their central counterparts because the morphology of the central cardiovascular signals is distorted at distal locations due to the transmission and reflection effects within the cardiovasculature intervening between the core and the periphery.

Techniques, to date, for inferring the dynamic relationship between core and peripheral signals by modeling of the intervening cardiovasculature, have required either multiple measurement points or else nominal model parameter values that are derived from prior experimentation, typically, on a population sample that is not necessarily representative of the individual on whom a given measurement is to be performed. Other techniques, such as described by Kamm et al., U.S. Pat. No. 6,117,087, while employing personalized parameters, require measurements at a substantial number of positions.

There have been several attempts to estimate the central aortic blood pressure by measuring and processing a single peripheral blood pressure: the generalized transfer function (GTF) technique, the method proposed by Sugimachi et al. (Japanese Journal of Physiology, vol. 51, pp. 217-222, (2001)), and the adaptive transfer function method proposed by Swamy et al. (Proc. IEEE Engineering in Medicine and Biology Conference, pp. 1385-1388, (2008)).

The GTF transfer function (GTF) technique, described by Gallagher et al., American Journal of Hypertension, vol. 17, pp. 1059-1067, (2004), is arguably the best accepted method in current clinical practice. In the GTF technique, a group-averaged relationship (that is identified from population-based experimentation) between the aortic and peripheral (usually upper limb) blood pressures processes the peripheral blood pressure measurement of a subject to estimate the aortic blood pressure. The GTF technique is not subject-specific, because the aortic-peripheral relationship is a group-averaged relationship. When an individual patient has an arterial state that is significantly different from the group-average, this technique can yield suboptimal results, as apparent from Stok et al., Journal of Applied Physiology, vol. 101, pp. 1207-1214, (2006), and Hope et al., J. Hypertension, vol. 21, pp. 1299-1305, (2003).

The method proposed by Sugimachi et al. uses a second physical measurement to improve the estimation of the central aortic blood pressure by measuring a single peripheral blood pressure: a non-invasively measured pulse delay time from aorta to the peripheral blood pressure measurement site is used in constructing the aortic-peripheral blood pressure relationship. The use of this additional information, the pulse delay time, may improve the accuracy of aortic blood pressure estimation. However, there are two drawbacks in this method: 1) (problem #1) except for the pulse delay time, the aortic-peripheral blood pressure relationship thus obtained still depends on the population-based parameters, and 2) (problem #2) it necessitates a second pulse measurement (the delay time) which was not needed in the GTF technique.

Swamy et al. resolved problem #1 of Sugimachi et al.'s method above, but did not solve problem #2. Swamy et al. proposed a method to estimate the parameters of the aortic-peripheral blood pressure relationship (except the pulse delay time) directly from the peripheral blood pressure measurement. Together with the direct measurement of pulse delay time, the aortic-peripheral blood pressure relationship they constructed is a truly subject-specific one, in the sense that all the parameters in the relationship belong to the specific subject: the pulse delay time is measured from the subject, and the other parameters are derived from the peripheral blood pressure measurement of the subject. However, this method could not solve the problem #2 of Sugimachi et al.'s method.

Each of the above methods has its own shortcomings: the GTF technique suffers form its lack of subject-adaptive capability, whereas the methods of Sugimachi et al. and Swamy et al. introduce an additional measurement modality for implementation.

Accordingly, it is highly desirable that cardiovascular wave propagation dynamics be identified without recourse to a priori knowledge relating to an exogenous population sample and without requiring an additional measurement modality.

### SUMMARY OF INVENTION

In accordance with preferred embodiments of the present invention, a method is provided for using a circulatory measurement on an extremity of a particular subject to derive an aortic blood pressure. The method, in a basic form, has steps of:

constructing a model that maps a peripheral cardiovascular waveform (CW) to a central cardiovascular waveform on the basis of a plurality of model parameters;

acquiring a time record of the peripheral CW with a non-invasive blood pressure sensor disposed at a solitary peripheral point;

transforming the time record of the peripheral CW to obtain a plurality of test central blood pressure waves, one test central blood pressure wave based on each of a plurality of sets of values of the model parameters;

electing an optimum set of values of the model parameters based on a specified criterion applied to the plurality of test central blood pressure waves; and

obtaining the aortic circulatory waveform of the subject based on the elected set of values of the model parameters.

In accordance with other embodiments of the invention, the elected set of values of the model parameters is specific to the particular subject. Each of the plurality of sets of values may correspond to successive putative transit times between the central CW and the peripheral CW. Electing an optimum set of values of the model parameters may, more specifically, include electing an optimum system order corresponding to an optimum transit time. Each putative transit time between the central CW and the peripheral CW may correspond to an order of a generalized auto-regressive moving average (ARMA) model.

In order to elect an optimum set of model parameter values, the specified criterion applied may be a criterion applied to the plurality of test blood pressure waveforms in the time domain, and, more particularly, the criterion may be a minimum norm of a second time derivative of the test central blood pressure wave. In certain embodiments of the invention, the model applied is an affine model.

In accordance with another aspect of the present invention, a computer program product is provided for use on a computer system for establishing an aortic circulatory waveform of a subject. The computer program product has a computer usable medium that contains computer readable program code, the computer readable program code including:

memory for storing a model that maps a peripheral cardiovascular waveform (CW) at a peripheral point to a central cardiovascular waveform on the basis of a plurality of model parameters;

an input for receiving a time record of a peripheral CW measured by a non-invasive blood pressure sensor;

a module for calculating a succession of sets of values for each of the plurality of model parameters;

computer code for transforming the time record of the peripheral CW to obtain a plurality of test central blood pressure waves, one test central blood pressure wave based on each of the set of model parameters;

a software module for selecting an optimum system order corresponding to an optimum transit time based on a specified criterion applied to the plurality of test central blood pressure waves; and

an output for displaying the aortic circulatory waveform of the subject based on the elected optimum system order and its corresponding model parameters.

### BRIEF DESCRIPTION OF THE DRAWINGS

Advantages of the present invention and its several improvements will be seen when the following detailed description is read in conjunction with the attached drawings. These drawings are intended to provide a better understanding of the present invention, but they are in no way intended to limit the scope of the invention.

FIG. 1 shows a non-invasive single-sensor central cardiovascular monitoring set-up, in accordance with an embodiment of the invention;

FIG. 2 depicts a single-segment transmission line model for aortic-to-radial blood pressure wave propagation;

FIG. 3 depicts examples of incident and reflecting blood pressure waves within the aortic-to-upper limb channel, as modeled in accordance with embodiments of the present invention;

FIG. 4 shows blood pressure signals for different values of n_{X}, and second derivative norms of the blood pressure signals for the respective values of n_{X};

FIG. 5(a) shows blood pressure signals for different values of n_{X}, while FIG. 5(b) shows second derivative norms of the blood pressure signals for the respective values of n_{X};

FIG. 6 shows an experimental plot of the recovered aortic blood pressure (BP) (solid curve) of a swine subject for a measured true radial BP (dotted curve) and true aortic BP (dashed curve); and

FIG. 7 compares true vs. reconstructed aortic blood pressure signals for a simulated human subject.

### DETAILED DESCRIPTION OF PREFERRED EMBODIMENTS

In accordance with preferred embodiments of the present invention, a novel method is provided for obtaining an input circulatory waveform, such as an aortic blood pressure (BP) signal, based on a measurement performed with a single non-invasive sensor applied at a point on the periphery of the cardiovascular system of a patient.

Instead of directly measuring pulse delay time, a spectrum of different, possible delays are analyzed. Then, an evaluation algorithm is used to judge which of these possible delays is most physiologically valid. The method also estimates all other parameters of the aortic-peripheral blood pressure relationship. As a result, the current invention is distinct from, and provides advantages relative to, all of the prior art methods, particularly in that it is capable of deriving subject-specific aortic-peripheral blood pressure relationship based only on a non-invasive, peripheral blood pressure measurement; this method eliminates the need to 1) use population-based parameters in the relationship, and 2) obtain pulse delay time measurement. In addition, this novel method exploits both systolic and diastolic portions of the data to derive the subject-specific parameters in the aortic-peripheral blood pressure relationship, while the method of Swamy et al., for example, uses only the diastolic portion of the data.

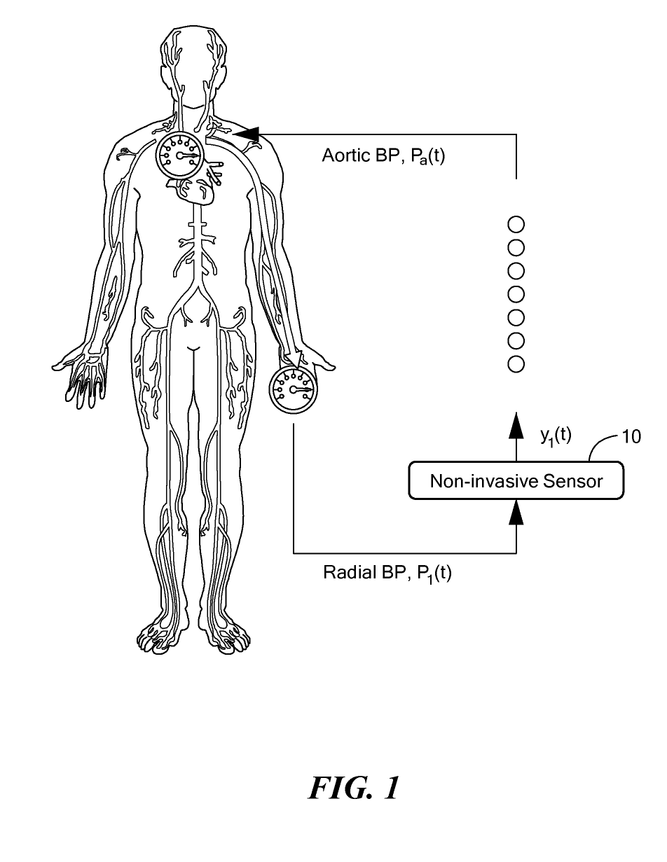

A “non-invasive” sensor 10 (shown in FIG. 1) is a sensor that does not penetrate through, or by way of, the skin. In particular, the present invention teaches a method for applying a temporal 2-channel blind identification methodology to a single-sensor monitoring modality.

Further description of the present invention is provided in Hahn, A System Identification Approach to Non-Invasive Central Cardiovascular Monitoring, Ph.D. Dissertation, Massachusetts Institute of Technology, (2008), which is incorporated herein by reference in its entirety.

Methods in accordance with the present invention posit a model describing the cardiovascular wave propagation dynamics. The use of any model mapping blood pressure waveforms from the core of the body to waveforms measured at the periphery of the circulatory system is within the scope of the present invention. For purposes of the present description and as used in any appended claims, the periphery of the circulatory system will refer to any region of blood flow within the body that is accessible to non-intrusive measurement of blood volume or pressure. When reference is made herein to a blood pressure sensor being disposed at a “point,” the usage is non-limiting, i.e., the sensor may extend over multiple points, however, all are external to the body of the subject, such that the sensor is applied non-invasively. Thus, blood flow that may be sensed from the surface of the body, or from beyond the surface of the body, constitutes peripheral blood flow. Examples that are of particular clinical utility encompass blood flow within a digital artery, or within the radial or ulnar artery, etc. However, these examples of peripheral blood flow are enumerated here without limitation. For convenience of explication, the peripheral blood pressure signal may be referred to herein, albeit without limitation, as an “upper-limb” blood pressure signal.

While the use of any model to describe the propagation of a circulatory waveform from the core of a patient to vasculature in the patient's periphery is encompassed within the scope of the present invention, preferred models entail an affine mapping between the space of temporal functions representing waveforms in the patient's core and temporal functions representing waveforms in the patient's periphery. As used herein and in any appended claims, the term “affine” refers to a mapping that is a linear transformation, where the mapping is from the space of aortic waveform functions into the space of waveforms as measured. The local affine relationship of the non-invasive sensor is not a strict restriction of the method, however, and the method described herein, and claimed in any appended claims, is advantageously applicable to many blood pressure monitoring devices that have an approximately affine relationship locally, or even globally, i.e., over their entire range.

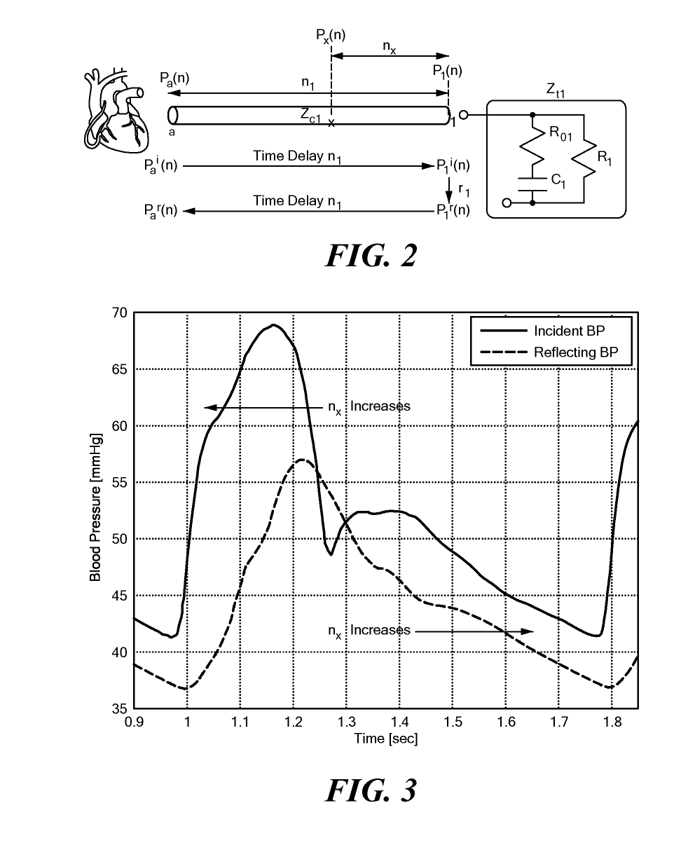

A preferred model for use in the method of the present invention is a single-segment transmission line model, an example of which is depicted in FIG. 2. The model relates the waveform of blood pressure waves at the origin, at the termination (modeled by its electrical analog) and at a location X along the transmission line. Assuming that the compliance and the inertance of the arterial vessel are both constant, and that any energy loss due to the visco-elasticity of the arterial vessel is negligible, then the blood pressure wave at location X in the aortic-to-radial CV system may be decomposed into its incident (forward-propagating) and reflecting (backward propagating) components:

P_{X}(t) [symbol] P_{X}ⁱ(t)+P_{X}^{r}(t), (Eqn. 1)

where

$$t {=}^{Δ} \frac{n}{{F}_{s}} ,$$

F_{S} is the sampling frequency, and n is the discrete transit time in units of sampling interval.

For heuristic purposes, a modified Windkessel terminal load impedance is used, as described by Liu et al., Impedance of arterial system simulated by viscoelastic tubes terminated in Windkessels, Am. J. Physiology, vol. 256, pp. 1087-99 (1989), which is incorporated herein by reference. In that case, the transfer function relating P_{X}(t) to P₁(t) becomes:

$${G}_{1} ( s ) {=}^{Δ} \frac{{P}_{1} ( s )}{{P}_{X} ( s )} = {e}^{- {τ}_{x} s} \frac{s + {a}_{1} + {b}_{1}}{s + {a}_{1} + {b}_{1} {e}^{- 2 {τ}_{X} s}} , \qquad ( Eqn . 2 )$$

where the parameters a₁ and b₁ may be expressed in terms of the Windkessel termination model, however, the specifics of the model are not essential to the invention as taught herein.

When cast in terms of discrete-time pressure wave propagation dynamics, the model constants a₁ and b₁, the transit time n_{X}, and the defined variable z [symbol] e^{s/F}^{ₛ}, the transfer function G₁(z) becomes:

$${G}_{1} ( z ) = \frac{{z}^{{n}_{X} + 1} + [ ( {a}_{1} + {b}_{1} ) / {F}_{s} - 1 ] {z}^{{n}_{X}}}{{z}^{2 {n}_{X} + 1} + ( {a}_{1} / {F}_{s} - 1 ) {z}^{2 {n}_{X}} + {b}_{1} / {F}_{s}} . \qquad ( Eqn . 3 )$$

Reverting to the time domain, expressions for the time series sequence of the upper-limb extremity blood pressure may be given as:

$${P}_{1} ( n + {n}_{X} + 1 ) = F [ {P}_{1} ( n + {n}_{X} ) , {P}_{1} ( n - {n}_{X} ) ] + {\sum }_{k = 1}^{2} {β}_{k} {P}_{X} ( n + 2 - k ) {=}^{Δ} [ 1 - \frac{{a}_{1}}{{F}_{s}} ] {P}_{1} ( n + {n}_{X} ) - \frac{{b}_{1}}{{F}_{s}} {P}_{1} ( n - {n}_{X} ) + {P}_{X} ( n + 1 ) - [ 1 - \frac{{a}_{1} + {b}_{1}}{{F}_{1}} ] {P}_{X} ( n ) , \\ where \\ {β}_{1} {=}^{Δ} 1 , {β}_{2} {=}^{Δ} - [ 1 - \frac{{a}_{1} + {b}_{1}}{{F}_{s}} ] , \\ and \\ F [ {P}_{1} ( n + {n}_{X} ) \cdot {P}_{1} ( n - {n}_{X} ) ] {=}^{Δ} [ 1 - \frac{{a}_{1}}{{F}_{s}} ] {P}_{1} ( n + {n}_{X} ) - \frac{{b}_{1}}{{F}_{s}} {P}_{1} ( n - {n}_{X} ) . \qquad ( Eqn . 4 )$$

After successive samplings, and taking the difference between them, one obtains:

$$Δ {P}_{1} ( n + {n}_{x} + 1 ) = Δ F [ Δ {P}_{1} ( n + {n}_{x} ) , Δ {P}_{1} ( n - {n}_{x} ) ] + {\sum }_{k = 1}^{2} {β}_{k} Δ {P}_{x} ( n + 2 - k ) , \\ where \\ Δ {P}_{1} ( n ) {=}^{Δ} {P}_{1} ( n + 1 ) - {P}_{1} ( n ) , Δ {P}_{X} ( n ) {=}^{Δ} {P}_{X} ( n + 1 ) - {P}_{X} ( n ) , \\ and \\ Δ F [ Δ {P}_{1} ( n + {n}_{X} ) , Δ {P}_{1} ( n - {n}_{X} ) ] {=}^{Δ} F [ {P}_{1} ( n + {n}_{X} + 1 ) , {P}_{1} ( n - {n}_{X} + 1 ) ] - F [ {P}_{1} ( n + {n}_{X} ) , {P}_{1} ( n - {n}_{X} ) ] = [ 1 - \frac{{a}_{1}}{{F}_{s}} ] Δ {P}_{1} ( n + {n}_{X} ) - \frac{{b}_{1}}{{F}_{s}} Δ {P}_{1} ( n - {n}_{X} ) \qquad ( Eqn . 5 )$$

Assuming that the input signal can be effectively approximated as a piecewise-constant signal over p≧3 sampling intervals, one obtains the following functional relationship which depends only on the upper-limb blood pressure signal:

$$Δ {P}_{1} ( n + {n}_{X} + 1 ) = [ 1 - \frac{{a}_{1}}{{F}_{s}} ] Δ {P}_{1} ( n + {n}_{X} ) - \frac{{b}_{1}}{{F}_{s}} Δ {P}_{1} ( n - {n}_{X} ) . \qquad ( Eqn . 6 )$$

If the blood pressure signal measured non-invasively is affine in the true extremity blood pressure signal, the Eqn. 6 can be written in the following linear regression in terms of the non-invasive blood pressure measurement y₁(n) instead of the true (i.e., upper-limb) blood pressure P₁(n):

$$Δ {y}_{1} ( n + {n}_{X} + 1 ) = [ 1 - \frac{{a}_{1}}{{F}_{s}} ] Δ {y}_{1} ( n + {n}_{X} ) - \frac{{b}_{1}}{{F}_{s}} Δ {y}_{1} ( n - {n}_{X} ) {=}^{Δ} {θ}^{T} ϕ ( n ) , \\ where \\ θ = [ \begin{matrix}{θ}_{1} \\ {θ}_{2}\end{matrix} ] {=}^{Δ} [ \begin{matrix}1 - \frac{{a}_{1}}{{F}_{s}} \\ - \frac{{b}_{1}}{{F}_{s}}\end{matrix} ] \\ and \\ ϕ ( n ) {=}^{Δ} [ \begin{matrix}Δ {y}_{1} ( n + {n}_{X} ) \\ Δ {y}_{1} ( n - {n}_{X} )\end{matrix} ] . \qquad ( Eqn . 7 )$$

Eqn. 7 is readily solved using least-squares techniques, thus derivation of the true aortic blood pressure requires a good estimate of the model order n_{X}. Obtaining that estimate of model order n_{X} is now described in accordance with preferred embodiments of the present invention.

First, it is known that the effect of the blood pressure wave reflection is to redistribute the blood pressure with the value of the mean blood pressure preserved, since it is totally an oscillatory phenomenon. Therefore, instantaneous change in the blood pressure due to reflection must average zero throughout the heart beat, which implies that if the superposition of incident and reflecting blood pressure waves is constructive at some portion of the heart beat, then it should be destructive at some other portion of the heart beat in such a way that the constructive and the destructive superposition is balanced out.

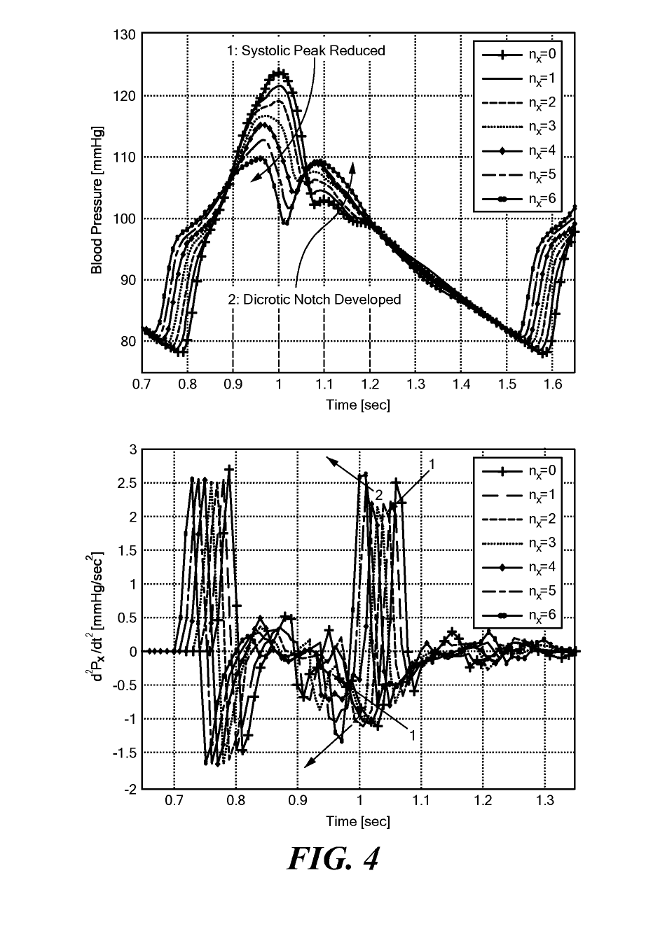

FIG. 3 illustrates an example of the incident and reflecting BP waves at the extremity location of the radial artery, i.e., n_{X}=0, which implies that the radial extremity BP is obtained by superimposing them with each other. The relationship between P₁(n) and P_{X}(n) suggests that, as the location X moves proximally towards the aorta (i.e., as n_{X} increases), the incident and reflecting components of the BP wave are separated by 2n_{X} in time, as shown in FIG. 3, before they are superimposed with each other to yield the BP wave P_{X}(n).

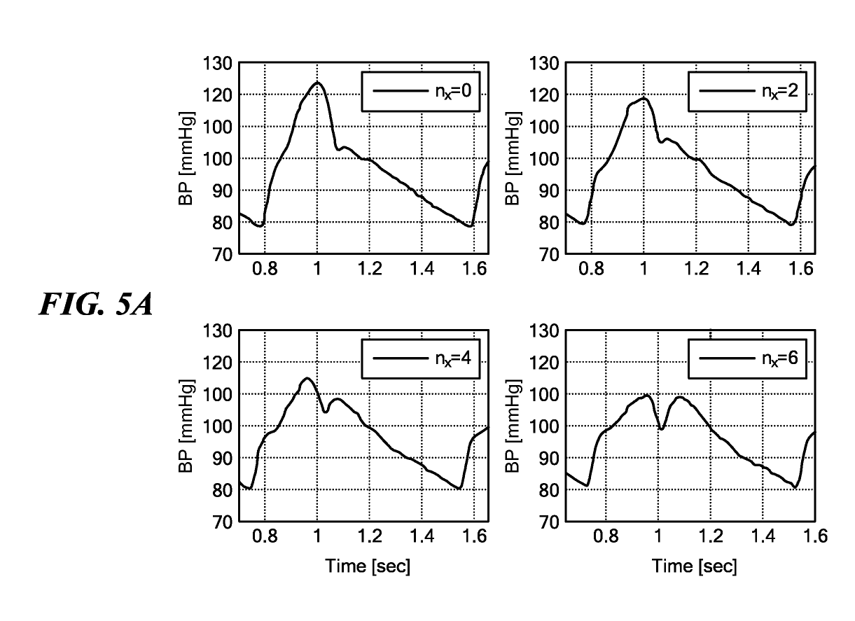

FIG. 4 shows an example of the BP signals P_{X}(n) that result from the incident and reflecting BP waves in FIG. 3, and their second derivatives for different values of n_{X}, i.e., n_{X}=0, . . . , 6, which correspond to 0, 10, . . . , and 60 ms of pulse travel times. In the example shown, the ascending aorta corresponds to n_{X}=4, and the blood pressures associated with n_{X}>4 are physically meaningless.

FIG. 4 demonstrates that:

1. For nₓ=0, the incident and the reflecting blood pressure waves superimpose in such a way that their superposition is highly constructive in systole, resulting in the largest pulse pressure at nₓ=0.

2. The systolic superposition of the incident and the reflecting BP waves becomes less constructive (see ‘1’ in FIG. 3) as n_{X} increases, which results in the reduction of the amplitude of the first peak (i.e., the systolic blood pressure).

3. The dicrotic notch starts to develop as n_{X} further increases so that the peaks of the incident and the reflecting BP waves are completely separated from each other, which also results in the “amplification” of the second peak (see ‘2’ in FIG. 3).

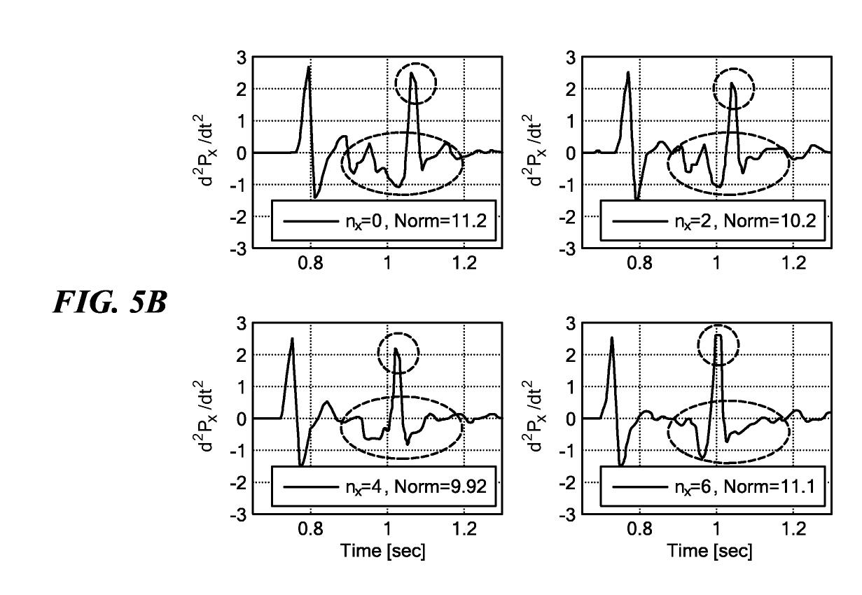

FIGS. 4 and 5 also illustrate that the fluctuation in the second derivative of the blood pressure wave heavily depends on the level of systolic blood pressure as well as the sharpness of the dicrotic notch. In particular, it is proportional to both of them. In terms of the second derivative norm of the blood pressure signal, therefore, it first decreases as the first peak is lowered in response to the increase in n_{X}, up to the value of n_{X} approximately corresponding to the ascending aortic location, where the dicrotic notch starts to develop. However, the dicrotic notch cancels out and even overwhelms the effect of lowering systolic blood pressure as n_{X} further increases, resulting in the increases in the second derivative norm as n_{X} exceeds the aortic-to-upper-limb pulse transit time.

Based on the analysis, therefore, the optimal estimate of n_{X}, i.e., the value of nₓ corresponding to the aortic-to-upper-limb pulse travel time, can be determined by identifying the value of n_{X} which minimizes the second derivative norm of the blood pressure signal over n_{X}. Noting that the error e(n) associated with the least-squares problem converges to the second derivative norm of the blood pressure signal as the sampling frequency increases, i.e.,

$$\begin{matrix}{lim}_{{F}_{s} \to ∞} e ( n ) = {lim}_{{F}_{s} \to ∞} \frac{1}{{n}_{1}} [ Δ {P}_{X} ( n + 1 ) - ( 1 - \frac{{a}_{1} + {b}_{1}}{{F}_{s}} ) Δ {P}_{X} ( n ) ] \\ = {F}_{s}^{2} \frac{{d}^{2} {P}_{X} ( t )}{d {t}^{2}} ,\end{matrix} \qquad ( Eqn . 8 )$$

the estimation of n_{X} can be combined with the least squares estimation of θ simply by increasing the sampling frequency; then, n_{X} can be estimated by minimizing the least squares error as a function of n_{X} if the sampling frequency is sufficiently high.

In order to demonstrate the improvement in accuracy of estimating the central aortic blood pressure by using the subject-specific relationship between the central aortic and the extremity blood pressures rather than the population-based relationship, the method in this invention was applied to 1) the experimental data obtained from swine subjects and 2) blood pressure signals of eight human subjects created by a full-scale human cardiovascular simulator, the results of which are described below. As used herein, the term “subject” typically refers to a human subject, although the invention is not so limited.

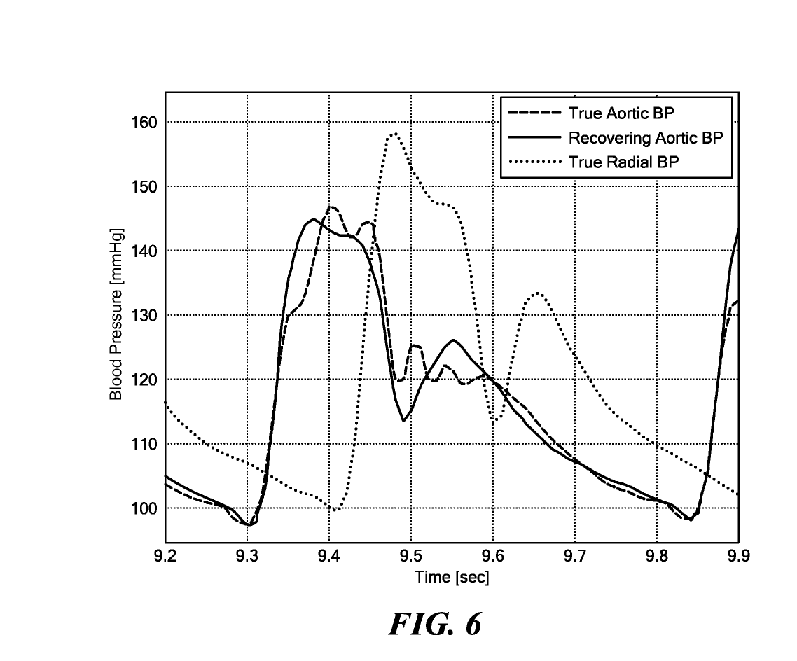

Methods in accordance with the present invention were tested in swine subjects and were found to be extremely accurate: using a peripheral blood pressure measurement, it was possible to estimate key features of aortic blood pressure with notable accuracy. In a study of 80 data segments taken in a swine subject under widely diverse physiologic conditions (e.g., during high and low blood pressures, fast and slow heart rates, vaso-constriction and vaso-dilation, etc.), it was found that, on average, the aortic systolic blood pressure was estimated within 0.4+/−5.0 mmHg, and the ejection duration was estimated within 0+/−0.01s. Direct invasive measurements of aortic blood pressure served as the gold standard against which the new technique was compared. An exemplary result from the experimental swine data is shown in FIG. 6.

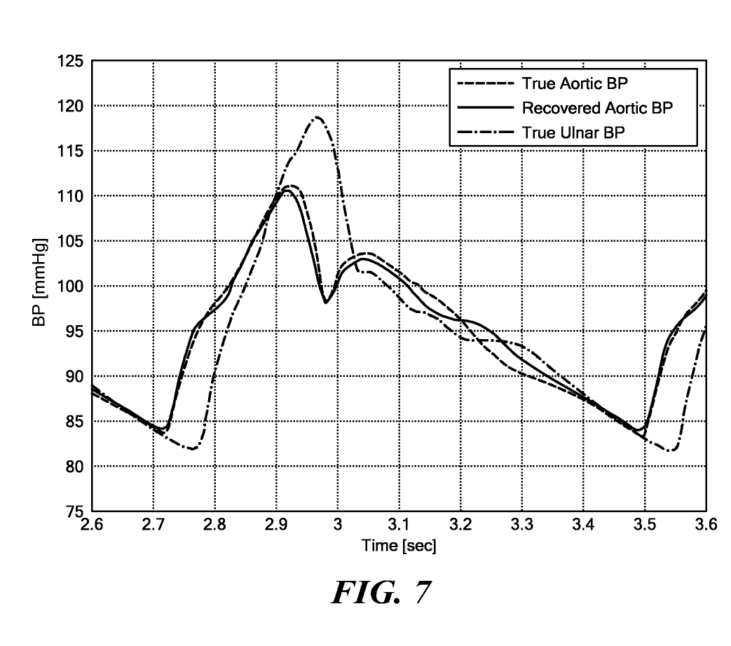

Data based on eight simulated human subjects, characterized by different physiologic states, are presented in FIG. 7. Using the peripheral blood pressure from the simulated subjects, the present invention accurately predicts the simulated aortic blood pressure.

In alternative embodiments, the disclosed methods for deriving data for the estimation of an aortic blood pressure waveform may be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

The described embodiments of the invention are intended to be merely exemplary and numerous variations and modifications will be apparent to those skilled in the art. In particular, blood characteristics other than arterial blood pressure may be measured employing the techniques described herein and is within the scope of the present invention. All such variations and modifications are intended to be within the scope of the present invention as defined in the appended claims.

We claim:

1. A method for deriving an aortic circulatory waveform of a particular subject, the method comprising:
constructing a model that maps a peripheral cardiovascular waveform (CW) to a central cardiovascular waveform on the basis of a plurality of model parameters;
acquiring a time record of the peripheral CW with a non-invasive blood pressure sensor disposed at a solitary peripheral point;
transforming the time record of the peripheral CW to obtain a plurality of test central blood pressure waves, one test central blood pressure wave based on each of a plurality of sets of values of the model parameters;
electing an optimum set of values of the model parameters based on a specified criterion applied to the plurality of test central blood pressure waves; and
obtaining the aortic circulatory waveform of the subject based on the elected set of values of the model parameters.

2. The method of claim 1, wherein the elected set of values of the model parameters is specific to the particular subject.

3. The method of claim 1, wherein each of the plurality of sets of values corresponds to successive putative transit times between the central CW and the peripheral CW.

4. The method of claim 3, wherein electing an optimum set of values of the model parameters includes electing an optimum system order corresponding to an optimum transit time.

5. The method of claim 3, wherein each putative transit time between the central CW and the peripheral CW corresponds to an order of a generalized auto-regressive moving average (ARMA) model.

6. The method of claim 1, wherein the specified criterion is a criterion applied to the plurality of test central blood pressure waves in the time domain.

7. The method of claim 6, wherein the specified criterion is a minimum norm of a second time derivative of the test central blood pressure wave.

8. The method of claim 1, wherein the model is an affine model.

9. A computer program product for use on a computer system for establishing an aortic circulatory waveform of a subject, the computer program product comprising a computer usable medium having computer readable program code thereon, the computer readable program code including:
memory for storing a model that maps a peripheral cardiovascular waveform (CW) at a peripheral point to a central cardiovascular waveform on the basis of a plurality of model parameters;
an input for receiving a time record of a peripheral CW from a non-invasive blood pressure sensor;
a module for calculating a succession of sets of values for each of the plurality of model parameters;
computer code for transforming the time record of the peripheral CW to obtain a plurality of test central blood pressure waves, one test central blood pressure wave based on each of the set of model parameters;
a software module for selecting an optimum system order corresponding to an optimum transit time based on a specified criterion applied to the plurality of test central blood pressure waves; and
an output for displaying the aortic circulatory waveform of the subject based on the elected optimum system order and its corresponding model parameters.

10. The computer program product of claim 9, wherein each of the plurality of sets of values corresponds to successive putative transit times between the central CW and the peripheral CW.

11. The computer program product of claim 10, wherein electing an optimum set of values of the model parameters includes electing an optimum system order corresponding to an optimum transit time.

12. The computer program product of claim 11, wherein each putative transit time between the central CW and the peripheral CW corresponds to an order of a generalized auto-regressive moving average (ARMA) model.

13. The computer program product of claim 10, wherein the specified criterion is a criterion applied to the plurality of test central blood pressure waves in the time domain.

14. The computer program product of claim 13, wherein the specified criterion is a minimum norm of a second time derivative of the test central blood pressure wave.

15. The computer program product of claim 10, wherein the model is an affine model.
